(19) United States
(12) Patent Application Publication
(10) Pub. No.: US 2008/0119862 A1
(43) Pub. Date: May 22, 2008

(54) Surgical Instrument for Supplying a Counter-Torque When Securing a Spinal Prosthesis

(76) Inventors: MeLeah Ann Wicker, Arlington, TN (US); Eric C. Lange, Collierville, TN (US); Henry Keith Bonin, Memphis, TN (US); John Durward Pond, Germantown, TN (US)

Correspondence Address: COATS & BENNETT/MEDTRONICS, 1400 CRESCENT GREEN, SUITE 300, CARY, NC, 27518, US

(21) Appl. No.: 11/562,238

(22) Filed: Nov. 21, 2006

(51) Int. Cl. A61B 17/58

(52) U.S. Cl. 606/99

(57) ABSTRACT

A surgical instrument for assembling a spinal prosthesis to a plurality of anchoring members may be used to guide a driving tool and, if desired, to apply a distraction or compression force. The instrument includes two guide tubes with respective proximal, distal, and intermediate sections. The guide tubes are pivotally connected at their intermediate sections so that their respective longitudinal axes intersect and the cannulations of the guide tubes intersect. The surgical instrument may include a locking mechanism connecting the proximal portions and selectively operative to preserve an angular relationship between the guide tubes against at least one of increasing or decreasing.

### BACKGROUND

The invention relates to a instrument and related method for securing a spinal prostheses during a surgical procedure.

Spinal implants are often inserted into a patient's body in order to stabilize an internal structure, promote healing, or relieve pain. For example, a common procedure involves the use of anchoring members, such as pedicle screws or hooks, joined by a flexible or rigid spinal rod in order to secure vertebrae in a desired position. Once the spinal rod is placed in the patient's body, the spinal rod should be firmly secured to the relevant anchoring members. Typically, this securing is achieved by rotating a set screw or other locking element to clamp the spinal rod, directly or indirectly, against the relevant anchoring element. However, the application of the necessary rotational force to the locking element tends to likewise apply an undesirable rotational force to the anchoring element. As such, some surgical methods involve the use of a guide tube that couples to the anchoring element. A driving tool is inserted through the guide tube and mates with the locking element. Then, when the tightening torque is applied to the locking element, the guide tube provides a means of applying a suitable counter-torque to the anchoring element.

Further, a surgeon often desires to distract or compress the relevant vertebrae when the spinal rod is secured in place, so that the spinal rod may help hold the vertebrae in a desired position. Typically, this is achieved by using a separate surgical distraction or compression instrument that must access the surgical site while the spinal rod is being secured as described above. The use of the separate distraction and/or compression tool may present complications during the spinal rod securing process.

While a number of specialized tools have been developed to facilitate the placement of spinal prostheses, including guide tubes and separate distractor/compressor tools, there remains a need for alternative surgical instrumentation, advantageously surgical instrumentation that is well suited to use during minimally invasive procedures.

### SUMMARY

In one illustrative embodiment, a surgical instrument for assembling a spinal prosthesis to a plurality of anchoring members comprises: a first guide tube having a first proximal end section, a first distal end section, and a first intermediate section, and extending along a first longitudinal axis; a second guide tube having a second proximal end section, a second distal end section, and a second intermediate section, and extending along a second longitudinal axis; the first and second guide tubes pivotally connected at the first and second intermediate sections so that the first and second longitudinal axes intersect. The surgical instrument may further comprise a locking mechanism connecting the proximal portions and selectively operative to preserve an angular relationship between the first and second guide tubes against at least one of increasing or decreasing. In some embodiments, the surgical instrument may be used to both guide a driving tool and to apply a distraction or compression force. Various aspects and embodiments are disclosed, which may be used alone or in any combination. Further, methods of using the surgical instrument embodiments are disclosed.

### BRIEF DESCRIPTION OF THE DRAWINGS

FIG. 1 shows a spinal prosthesis and associated bone screw assemblies partially installed on vertebrae.

FIG. 2 shows a perspective view of a surgical instrument according to one embodiment of the present invention.

FIG. 3 shows an upper portion of the surgical instrument of FIG. 2.

FIG. 4 shows the surgical instrument of FIG. 2 about to be mated to bone screw assemblies.

FIG. 5 shows the surgical instrument of FIG. 2 mated to bone screw assemblies.

FIG. 6 shows the surgical instrument of FIG. 2 in a spread and unlocked configuration.

FIG. 7 shows the surgical instrument of FIG. 2 in a spread and locked configuration.

FIG. 8 shows the surgical instrument of FIG. 2 with a driving tool inserted in one guide tube assembly.

FIG. 9 shows the surgical instrument of FIG. 2 with a driving tool inserted in another guide tube assembly.

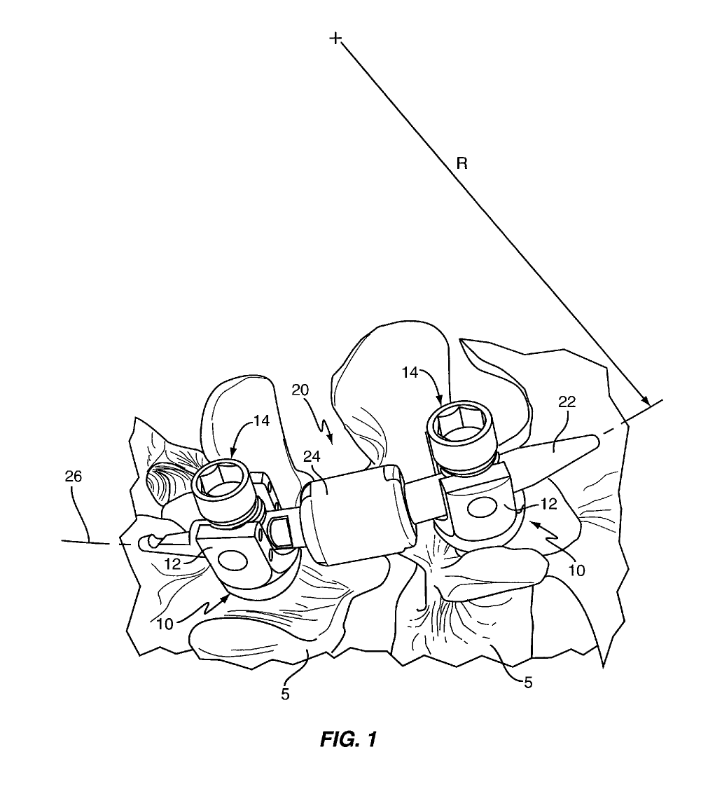

### DETAILED DESCRIPTION

Illustrative embodiments of the present invention include a surgical instrument and/or a method of using a surgical instrument in association with the insertion of a spinal prosthesis 20. One common example of such a spinal prosthesis 20 is a spinal rod. As such, the discussion below uses a spinal rod as an illustrative example of a spinal prosthesis 20. The particular spinal rod 20 used for illustrative purposes includes relatively rigid end sections 22 disposed on either side of a relatively flexible middle section 24. The spinal rod is generally elongate along a curvate longitudinal axis 26. The curve of the longitudinal axis is typically a continuous curve with a relatively constant radius of curvature R. The end sections 22 are typically mated to conventional polyaxial pedicle screw assemblies 10, which are in turn mated to the relevant vertebrae 5. The spinal rod 20 may be secured to the pedicle screws by clamping the rod 20 to the head 12 of the pedicle screw assembly via a set screw other locking element 14. For further information, attention is directed to U.S. Patent Application Publication 2005/0171540, which is incorporated herein by reference in its entirety. However, it should be understood that the present invention is not limited to use with the particular spinal rod 20 shown, and may instead be used with any suitable spinal prosthesis.

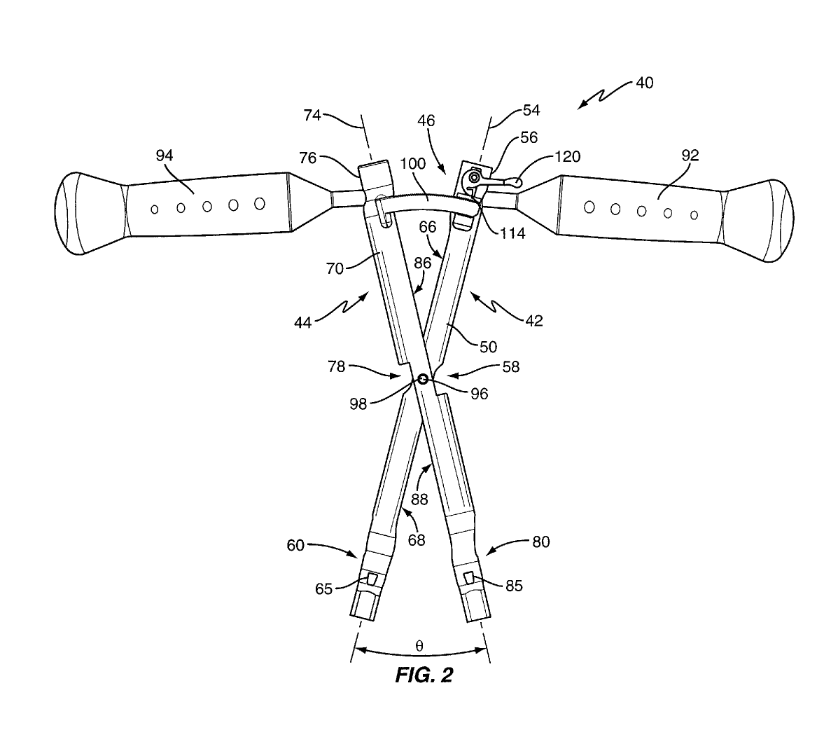

The instrument according to one embodiment is shown in FIG. 2, and generally indicated at 40. The instrument generally includes two guide tube assemblies 42,44 that are pivotally connected together, and a locking mechanism 46. The guide tube assemblies 42,44 provide a means for guiding a driving (or tightening) tool 30, and advantageously providing a counter-torque to the tightening torque, when securing a locking element 14 of a bone screw assembly 10. The tightening tool 30 is inserted through one guide tube assembly 42 or 44 to tighten a first locking element 14, removed, and then inserted into the other guide tube assembly 44 or 42 in order to tighten another locking element 14, without having to move the instrument 40. Further, the guide tubes 42,44 are pivotally connected, and are therefore also able to provide a distraction or compression force if desired. The locking mechanism 46 acts to help hold guide tubes 42,44 in a desired angular relationship when applying the distraction or compressive force.

The inner guide tube assembly 42 includes a guide tube 50 and a handle 92. The guide tube 50 is an elongate body extending along a longitudinal axis 54, with a central passage or cannulation 52 extending therethrough. The guide tube 50 includes a distal section 60, a proximal section 56, and an intermediate section 58. The distal section 60 may include an outer taper 62 if desired, and includes a generally U-shaped channel 64 that is disposed transverse to the longitudinal axis 54. Further, the distal section 60 may include suitable ports 65 for allowing the entry of light and/or allowing the interior of the central passage 52 in the distal section 60 to be viewed, as is desired. The proximal section 56 is disposed generally opposite the distal section 60, and advantageously includes a suitable mating section for mating with the corresponding handle 92. The intermediate section 58 is disposed between the distal section 60 and the proximal section 56. The outer wall of guide tube 50 includes an upper slot 66 that extends from the proximal section 56 to the intermediate section 58. This upper slot 66 advantageously extends through an arc of approximately 90° relative to the periphery of guide tube 50, and is disposed to face guide tube 70. The upper slot 66 terminates at a point below pivot point 96 where the two guide tube 50,70 are pivotally connected. Guide tube 50 further includes a lower slot 68, similar to the upper slot 66, but extending distally from the intermediate section 58 to the distal section 60. The lower slot 68 advantageously terminates proximate the taper 62 in distal section 60, if present. Handle 92 connects to proximal section 56 and extends outwardly and generally transverse thereto. If desired, the handle 92 may extend at a slight upward angle of approximately 10°. Handle 92 may take any form known the art, such as the generally elongate body with an elastomeric grip of increased size shown in FIG. 2. Handle 92 may be permanently mounted to guide tube 50, such as by being integrally formed or welded thereto, or may be removably mounted thereto, such as by a quick-connect mechanism (e.g., bayonet type connection), as is desired.

Guide tube assembly 44 is similar to guide tube assembly 42, but is of a larger diameter in the intermediate section 78, and has a slightly different slot structure. Thus, guide tube assembly 44 likewise includes a guide tube 70 and a handle 94. Guide tube 70 is an elongate body extending along a longitudinal axis 74, with a central passage or cannulation 72 extending therethrough. Guide tube 70 includes a distal section 80, a proximal section 76, and an intermediate section 78. As shown in FIG. 2, the distal section 80 may include an outer taper 82 if desired, and includes a generally U-shaped channel 84 that is disposed transverse to the longitudinal axis 74. Further, distal section 80 of guide tube 70 may include suitable ports 85 for allowing the entry of light and/or allowing the interior of the central passage 72 in the distal section 80 to be viewed, as is desired. The proximal section 76 is disposed generally opposite the distal section 80, and advantageously includes a suitable mating section for mating with the corresponding handle 94. The intermediate section 78 is disposed between the distal section 80 and the proximal section 76. The outer wall of guide tube 70 includes an upper slot 86 that extends from the proximal section 76 to the intermediate section 78. This upper slot 86 advantageously extends through an arc of >90° relative to the periphery of guide tube 70, and is disposed to face guide tube 50. Like upper slot 66, upper slot 86 terminates at a point below pivot point 96. Guide tube 70 further includes a lower slot 88, similar to the upper slot 86, but extending distally from intermediate section 78 to distal section 80. The lower slot 88 advantageously terminates proximate the taper 82 in distal section 80, if present. Both upper slot 86 and lower slot 88 should be wide enough to accommodate the inner guide tube 50. Further, the terminal portions of slots 86,88 near pivot point 96 may provide a mechanical stop to over-rotation of guide tube 50 relative to guide tube 70, or other means (e.g., locking mechanism 46) may be employed for this purpose. Handle 94 is substantially identical to handle 92, but connects to proximal section 76 rather than proximal section 56.

As can be seen in FIG. 2, guide tubes 50,70 are pivotally connected at their intermediate sections 58,78. In the pivot area, guide tube 50 passes through guide tube 70, with the result that the two guide tubes 50,70 form a X-shape and the respective cannulations 52,72 intersect. More particularly, guide tube 50 passes through upper slot 86 and lower slot 88 of guide tube 70 at an angle, so that the respective longitudinal axes form an included angle Φ. Because the two guide tubes 50,70 are pivotally connected, this included angle Φ is variable. Thus, angle Φ is relatively small when the distal portions 60,80 are disposed close together, and relatively larger when the distal sections 60,80 are disposed farther apart.

The pivoting action of guide tubes 50,70 may be achieved in a variety of ways. For example, intermediate section 58 of guide tube 50 may include a pair of outwardly extending stubs (not shown) that fit into corresponding holes in guide tube 70. The outer ends of these stubs may then be upset to join guide tube 50 to guide tube 70, while allowing for rotation about pivot axis 96. Of course, the male/female relationship may be reversed if desired. Alternatively, appropriate shoulder bolts 98 or short pivot pins may be employed. Advantageously, the bolts, pins, or other pivot means should be of such a length so as to not extend significantly into the central passage 52 of inner guide tube 50. For example, it may be advantageous to fuse weld the relevant bolt, pin, or other means flush with the interior surface of central passage 52.

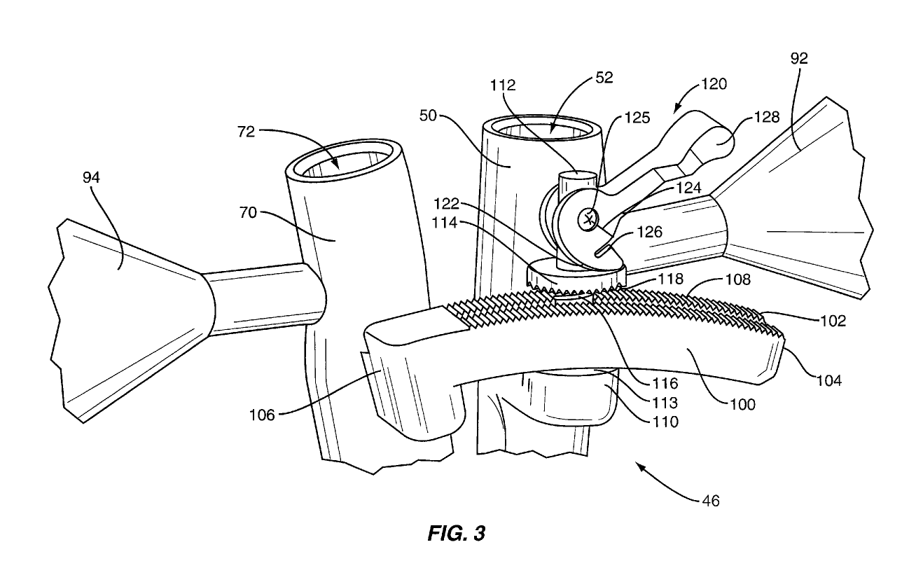

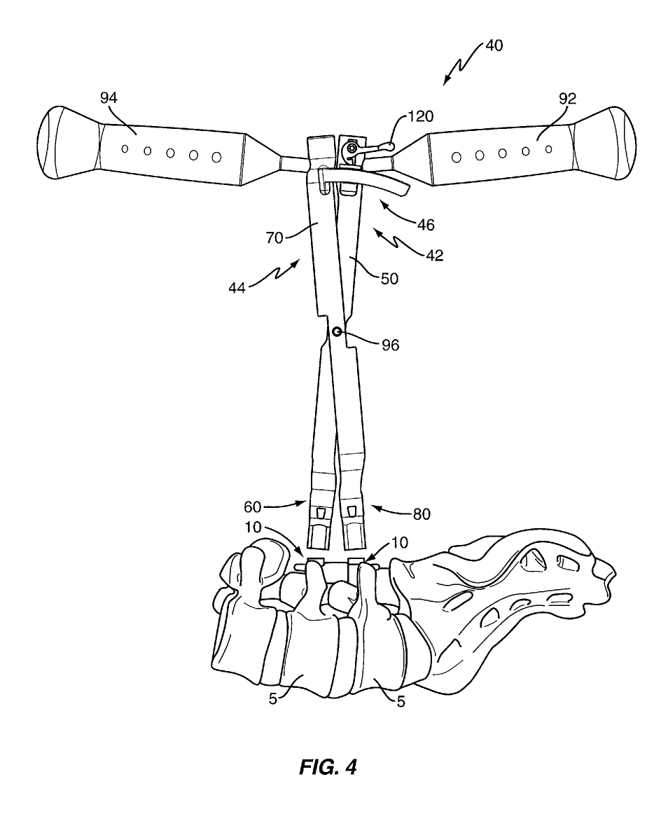

Locking mechanism 46 acts to help hold guide tubes 50,70 in a desired angular relationship. Locking mechanism 46 includes an arm 100, a support flange 110, a floating lock plate 114, and a locking lever 120. Arm 100 is mounted to guide tube 70 and extends toward guide tube 50. As can be seen in FIG. 2 arm 100 is advantageously curved with a radius of curvature centered at pivot point 96. If desired, the mounted end of arm 100 may be offset slightly from the sidewall of guide tube 70 by a suitable offsetting section 106, and arm 100 may be split at its outer extent, so as to form two fingers 102,104. The upper surface of arm 100 includes a plurality of projections (e.g., teeth) 108 for engaging with corresponding projections 118 on the underside of lock plate 114. Support flange 110 is mounted to, or is integrally formed with, guide tube 50. Support flange 110 extends laterally outward from guide tube 50 and provides support for lock plate 114 and locking lever 120. Support flange 110 may take any suitable form, but advantageously includes a post 112 extending upward from a surrounding platform area 113. Locking plate 114 is slidably disposed on post 112 so as to be moveable between a locked position and a release position. When in the locked position, protrusions (e.g., teeth) 118 on the locking plate's lower surface engage with corresponding projections 108 on arm 100 so as to lock the relative positions of guide tubes 50,70. Accordingly, locking plate 114 is advantageously biased, such as by spring 116, toward the release position, but is selectively forced to the locked position by locking lever 120. Locking lever 120 is mounted to post 112 so as to rotate about axis 124. Any means known in the art may be used to achieve this rotational mounting, such as by using a shoulder screw 125, pivot pin, or the like. Locking lever 120 includes a lever arm 128 and a curvate main body that forms a cam surface 122. Depending on the rotational position of locking lever, cam surface 122 engages locking plate 114 and forces locking plate 114 downward toward arm 100. If desired, locking lever 120 may include a relief 126, as shown in FIG. 3, that allows some give in cam surface 122 in order to enable an over-center type of locking action.

The instrument 40 may be used to secure a spinal rod 20 relative to anchor members 10, while applying a distraction force to the relevant vertebrae 5. Assuming the anchor members 10 to be conventional polyaxial bone screw assemblies, the bone screws are secured in place on the vertebrae 5 in a conventional fashion. Depending on the design of the polyaxial screw assembly, the head 12 or “tulip” of the assembly may or may not be locked down against polyaxial movement at this point. The spinal rod 20 is inserted into the heads 12 of the polyaxial screw assemblies 10 in a conventional fashion. The instrument 40 is then placed in position, with the distal end sections 60,80 engaging respective bone screw assemblies 10. During this portion of the installation process, the locking mechanism 46 is advantageously unlocked so as to allow the guide tubes 50,70 to be more easily positioned. Note that the channels 64,84 in distal sections 60,80 are advantageously configured to allow the respective guide tubes 50,70 to fit over the spinal rod 20 while engaging the heads 12 of the bone screw assemblies 10. Further, the interior of the central passages 52,72 in the tip portion of distal sections 60,80 may advantageously include suitable flattened areas (not shown), or other means known in the art, for non-rotatably mating with their respective bone screw assembly 10.

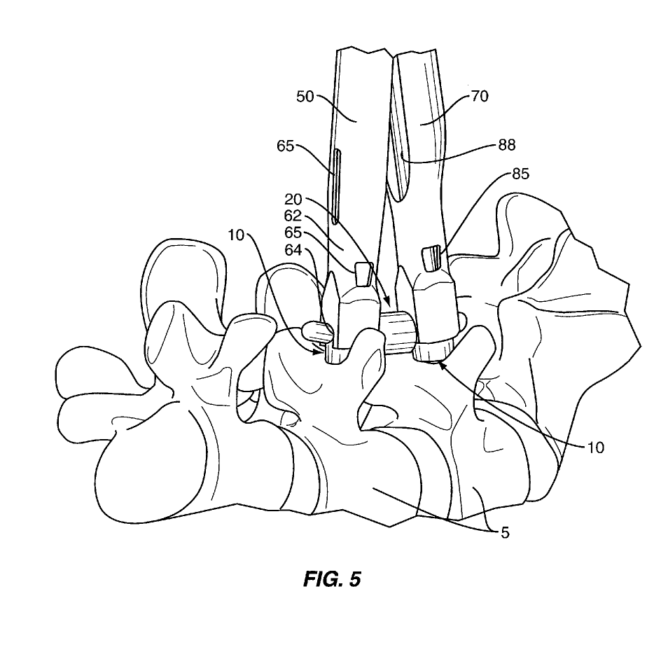

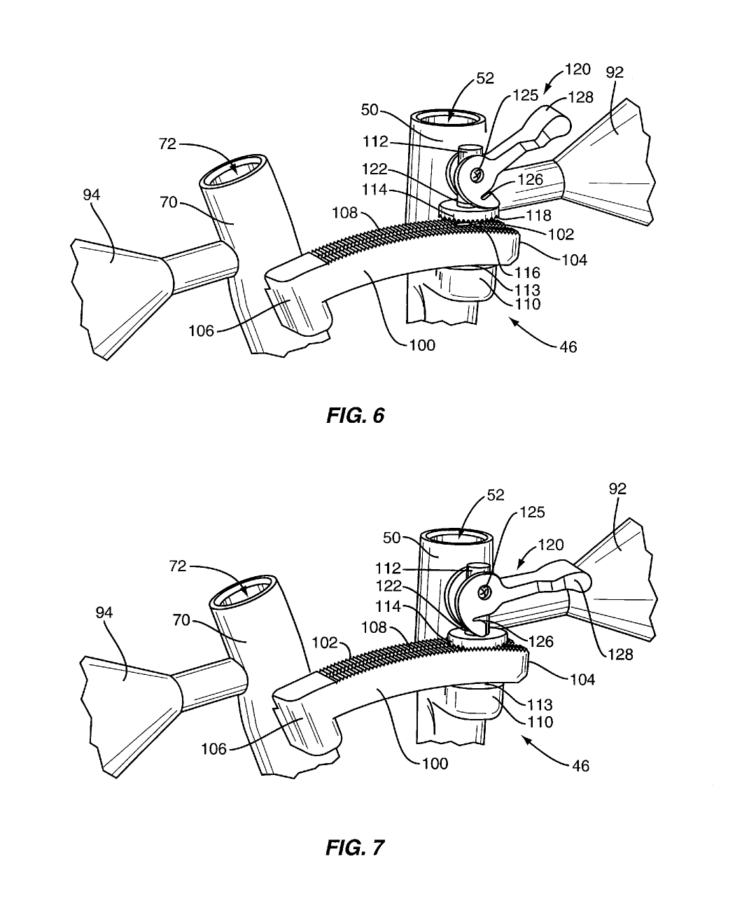

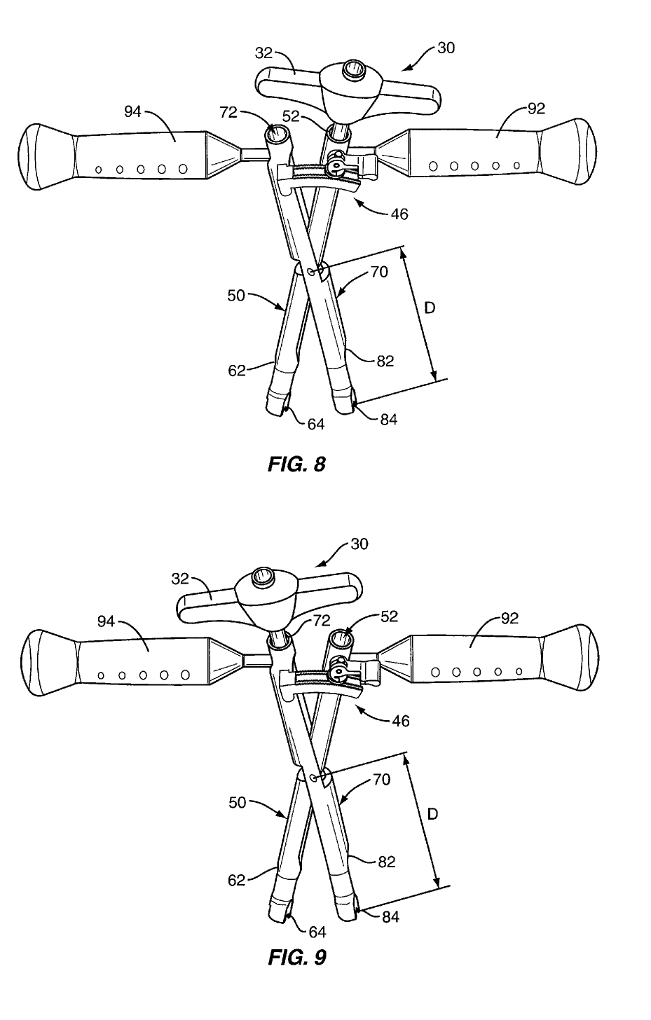

If not already present, handles 92,94 are then joined to their respective guide tubes 50,70. A distraction force is then applied to the vertebrae 5 by pulling the handles 92,94 apart. This has the effect of displacing the distal sections 60,80 of guide tubes 50,70 away from one another. When the desired level of distraction is achieved, the surgeon rotates locking lever 120 (clockwise in FIG. 6) by pressing on lever arm 128. This causes locking plate 114 to be forced toward arm 100 due to the cam action of cam surface 122. The protrusions 108,118 are brought into contact, and the locking lever 120 is held in the locked position by the over-center action provided by relief 126. It should be noted that the distraction is not linear, but is instead along an arc centered about pivot point 96. Advantageously, the radius of curvature of the distraction arc matches the radius of curvature R of the spinal rod's axis 26, and is centered about the same point 96. Thus, advantageously, the distance from the guide tube pivot point 96 to both of the respective channels 64,84, along respective axes 54,74, is approximately equal to the spinal rod's radius of curvature R.

With the instrument 40 in the locked position, a conventional driving tool 30 is inserted into one of the guide tubes 50 in order to secure the corresponding locking members 14 of the bone screw assemblies 10. Assuming that guide tube 50 is used first, the driving tool 30 extends down central passage 52, with a handle section 32 of the driving tool 30 exposed out the proximal section 54 of guide tube 50 for manipulation in the conventional fashion. It should be noted that the presence of the upper slot 86 and lower slot 88 in guide tube 70 allows the driving tool 30 to extend along the longitudinal axis 54 of guide tube 50, crossing over longitudinal axis 74 of guide tube 70, without being impeded. During the tightening process, a torque is applied to the locking member 14; a counter-torque is applied to the head 12 of the corresponding bone screw assembly 10 and/or to prosthesis 20, via their interaction with guide tube 50. Thus, the locking member 14 may be secured easily. When the locking member 14 is secured, driving tool 30 may be removed from guide tube 50 and inserted into guide tube 70 in order to tighten the locking member 14 of the other bone screw assembly 10. Note that this is achieved without having to relocate instrument 40. The second locking member 14 is then tightened, and the driving tool 30 removed from guide tube 70. With the spinal rod 20 now secured to the bone screw assemblies 10, the locking mechanism 46 may be released so that the distraction force is now provided by the spinal rod 20. The instrument 40 may then be removed from the surgical site, and the surgical process continue from this point in a conventional fashion.

The discussion above has assumed that a distraction force was to be applied to the vertebrae 5. However, the same instrument 40 may be used instead to apply a compressive force to the vertebrae 5 by pushing the guide tubes 50,70 together and then actuating locking mechanism 46. Indeed, the instrument 40 may also be used in situations where no distraction or compressive forces are to be applied, or where other instrumentation is used to generate such forces. Further, as pointed out above, the instrument 40 may be used with, inter alia, flexible spinal rods, pre-bent rigid spinal rods, and/or straight rigid spinal rods.

Because the guide tube assemblies 42,44 of the surgical instrument 40 of FIG. 2 cross and pivot at a non-varying pivot point 96, the instrument 40 of FIG. 2 allows for distraction/compression to occur along an arc, and provides meaningful tactile feedback to the surgeon regarding that amount of distraction/compression. Further, because the surgical instrument 40 mates to two spaced apart bone screw assemblies 10, the instrument 40 itself acts as an anti-rotation stabilizing element during locking member tightening. This stabilization helps relieve some undesirable loading on the spinal rod 20, particularly a flexible spinal rod, during locking member tightening.

The discussion above has also assumed that the locking mechanism 46 is operative to lock the relative positions of the guide tubes 50,70 in both directions. However, in some embodiments, it may be advantageous for the locking mechanism 46 to prevent relative motion of the guide tubes 50,70 in one direction (such as toward one another), while allowing for motion in the opposite direction. For such embodiments, a ratcheting locking mechanism 46 may be used, such as by appropriately configuring protrusions 108 and protrusions 118, or by using a rack/pawl ratcheting mechanism. For such embodiments, the locking mechanism 46 operates more as a retention mechanism than a true locking mechanism; however, such mechanisms are intended to be encompassed by the term “locking mechanism.”

The discussion above has assumed that handles 92,94 are connected to their respective guide tubes 50,70 in a single fixed orientation. However, in some embodiments the handles 92,94 may be connected to their respective guide tubes 50,70 so that the relative angle between the handles 92,94 and the respective guide tube's longitudinal axis 54,74 may be selected by a surgeon. For example a suitable variable angle lockable detent mechanism, of a type known in the art, may be used to interconnect the handles 92,94 with their respective guide tube proximal sections 56,76. The surgeon may then select the desired angle, such as 0°, 10°, 15°, 30°, 45°, 60°, 90°, and then lock the handle relative to the corresponding guide tube 50,70, and use the device as described above.

The discussion above has also assumed that the anchoring member is a bone screw assembly; however, the surgical instrument may likewise be used with other forms of anchoring members known in the art, such as hook assemblies and the like.

The various aspects of the surgical instrument 40, such as dual counter-torque action, locking distraction/compression, and other aspects, may be found individually in various embodiments of the surgical instrument 40, or in any combination. Further, while it is contemplated that the surgical instrument 40 may be advantageously used for installation of prosthesis 20 from a posterior approach, other approaches, such as an anterior, lateral, oblique, or any other surgical approach, may alternatively used.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A surgical instrument for assembling a spinal prosthesis to a plurality of anchoring members comprising:
a first guide tube having a first proximal end section, a first distal end section, and a first intermediate section, and extending along a first longitudinal axis;
a second guide tube having a second proximal end section, a second distal end section, and a second intermediate section, and extending along a second longitudinal axis;
said first and second guide tubes pivotally connected at said first and second intermediate sections so that said first and second longitudinal axes intersect.

2. The surgical instrument of claim 1 wherein said distal portions form an acute included angle.

3. The surgical instrument of claim 1 wherein said first and second guide tubes comprise respective first longitudinal openings extending proximally from said respective intermediate sections, said first openings facing each other.

4. The surgical instrument of claim 3 wherein said first and second guide tubes further comprise respective second longitudinal openings extending distally from said respective intermediate sections, said second openings facing each other.

5. The surgical instrument of claim 1 further comprising a locking mechanism connecting said proximal portions and selectively operative to preserve an angular relationship between said first and second guide tubes against at least one of increasing or decreasing.

6. The surgical instrument of claim 5 wherein said locking mechanism comprises an arcuate arm extending from said first proximal portion toward said second proximal portion.

7. The surgical instrument of claim 6 wherein said locking mechanism further comprises a moveable lock member associated with said second proximal portion and selectively engageable with said arcuate arm.

8. The surgical instrument of claim 7 wherein said lock member comprises a cam surface, said cam surface bearing against a moveable lock plate when said locking mechanism is in a locked position.

9. The surgical instrument of claim 8 wherein said arcuate arm includes a first set of projections, and wherein said lock plate comprises a second set of projections that interengage with said first set of projections when said locking mechanism is in said locked position.

10. The surgical instrument of claim 7 wherein said lock member is rotatable about an axis that is substantially perpendicular to said second longitudinal axis.

11. The surgical instrument of claim 1 further comprising a first handle mounted to said first proximal end section and a second handle mounted to said second proximal end section; said handles extending generally transverse to the longitudinal axis of the respective guide tube.

12. The surgical instrument of claim 5 wherein said locking mechanism is selectively operative to preserve said angular relationship between said first and second guide tubes against both of increasing and decreasing.

13. The surgical instrument of claim 1 further comprising a spinal prosthesis having a curvate longitudinal axis with a first radius of curvature; wherein said first and second distal end sections have respective transverse channels therein; wherein said first and second guide tubes pivotally connect at a point spaced from said transverse channels by a distance approximately equal to said first radius of curvature.

14. The surgical instrument of claim 1 wherein said first and second guide tubes comprise respective longitudinal passages passing therethrough; and wherein said longitudinal passages, in said distal sections, are configured to mate with a pedicle screw assembly.

15. The surgical instrument of claim 1 further comprising an elongate driver member disposed in an interior longitudinal passage of one of said first and second guide tubes.

16. A method of assembling a spinal prosthesis to a plurality of anchoring members comprising:
providing a surgical instrument comprising:
a first guide tube having a first proximal end section, a first distal end section, and a first intermediate section, and extending along a first longitudinal axis;
a second guide tube having a second proximal end section, a second distal end section, and a second intermediate section, and extending along a second longitudinal axis;
said first and second guide tubes pivotally connected at said first and second intermediate sections so that said first and second longitudinal axes intersect;
joining said first guide tube to a first anchoring member and said second guide tube to a second anchoring member;
securing a locking member of said first anchoring member by inserting a driving tool into a longitudinal passage of said first guide tube;
thereafter, removing said driving tool from said longitudinal passage of said first guide tube;
thereafter, securing a locking member of said second anchoring member by inserting said driving tool into a longitudinal passage of said second guide tube, without disjoining said first guide tube from said first anchoring member.

17. The method of claim 16 further comprising locking said guide tubes relative to one another to preserve an angular relationship between said first and second guide tubes against at least one of increasing or decreasing by causing a locking mechanism associated with said surgical instrument to assume a locked position.

18. The method of claim 17 wherein said locking mechanism remains in said locked position during said securing said locking member of said first anchoring member.

19. The method of claim 16 further comprising applying a distraction or compression force via said first and second guide tubes.

20. The method of claim 16 wherein said applying a distraction or compression force via said first and second guide tubes comprises applying said distraction or compression force along an arc via said first and second guide tubes.

21. The method of claim 20 wherein said arc has a first radius of curvature, and wherein a pivot point between said first and second guide tubes is disposed a distance from said first and second anchor member of approximately said first radius of curvature.

22. A surgical instrument for assembling a spinal prosthesis to a plurality of anchoring members comprising:
a first elongate hollow shaft having a first proximal end section, a first distal end section, and a first intermediate section, and extending along a first longitudinal axis; said first shaft having a first cannulation therethrough;
a second elongate hollow shaft having a second proximal end section, a second distal end section, and a second intermediate section, and extending along a second longitudinal axis; said second shaft having a second cannulation therethrough;
said first and second shafts pivotally connected at said first and second intermediate sections so that said first and second cannulations intersect.

23. The surgical instrument of claim 22 further comprising a locking mechanism connecting said proximal portions and selectively operative to preserve an angular relationship between said first and second shafts against at least one of increasing or decreasing; a first portion of said locking mechanism associated with said first shaft so as to move therewith and a second portion of said locking mechanism associated with said second shaft so as to move therewith.

24. The surgical instrument of claim 22 wherein said first portion comprises an arcuate arm extending from said first shaft toward said second shaft.

25. The surgical instrument of claim 24 wherein said arm, distal from said first shaft, comprises two generally parallel portions spaced from one another.

26. The surgical instrument of claim 22 wherein said first and second shafts pivot with respect to each other about a pivot axis fixed relative to said first and second shafts.
